(19) United States
(12) Patent Application Publication
(10) Pub. No.: US 2012/0183950 A1
(43) Pub. Date: Jul. 19, 2012

(54) TISSUE ANALYSIS

(76) Inventors: Benjamin-Leo Nicholas, Dorset (GB); Karl James Staples, Southampton (GB); Thomas Michael Alan Wilkinson, Hampshire (GB); Ratko Djukanovic, Southampton (GB)

(21) Appl. No.: 13/321,265

(22) PCT Filed: May 19, 2010
(86) PCT No.: PCT/GB10/50821

§ 371 Date: Apr. 6, 2012

(30) Foreign Application Priority Data
May 21, 2009 (GB) 0908788.3

(51) Int. Cl. G01N 33/53; C12Q 1/37; C12Q 1/70; C12Q 1/02

(52) U.S. Cl. 435/5; 435/29; 435/7.1; 435/7.2; 435/23; 435/7.24

(57) ABSTRACT

The present invention provides a method of tissue analysis, disease modelling or drug development comprising the steps of providing a tissue sample comprising cells of at least two different cell types; exposing cells in the tissue sample to an agent ex vivo; exposing the cells to three or more detection means arranged to distinguish between three or more different cell types and/or different cell states; detecting the detection means in order to determine the different cell types and/or different cell states in the sample. The invention further provides a kit for performing the method of the invention and uses of the method of the invention.

This invention relates to the field of tissue analysis, in particular a method of ex vivo tissue analysis, use of the method of tissue analysis and a kit for tissue analysis.

Drug development programmes currently rely on animal models of disease to assess efficacy and toxicity of new therapeutics before proceeding into expensive and potentially high risk human clinical trials. The use of these animal systems has often led to inappropriate selection of candidate compounds for clinical development resulting in high costs and adverse clinical consequences.

Asthma and chronic obstructive pulmonary disease (COPD) are common diseases. They are a major cause of ill-health and a large health-economic burden. The prevalence of both diseases is increasing and of particular worry is that, in contrast to other major diseases (e.g. cardiovascular), mortality from COPD continues to rise and is expected to be the third cause of death worldwide by 2020. Viruses account for up to 70% of respiratory infections in susceptible patients with these conditions and have the potential to cause significant morbidity and mortality (Mallia, P. and S. L. Johnston. 2006. Chest 130:1203-1210). Infectious exacerbations of asthma and COPD lead to increased airways inflammation that results clinically in acute symptoms such as reduced lung function, breathlessness and decline in quality of life measures. Furthermore, in COPD patients viral infections predispose to secondary bacterial infection (Wilkinson, T. M. et al. 2006. Chest 129:317-324) a phenomenon which may be due to increased bacterial binding to epithelium damaged by viruses (Hakansson, A. et al. 1994. Infect. Immun. 62:2707-2714).

Significant research effort has gone into establishing animal models of COPD which mimic the inflammatory processes in humans. This has required manipulation of the airways by such chronic stimuli as tobacco smoke (Maeno T, et al. J Immunol 2007; 178(12):8090-8096) or bacterial by-products such as LPS (Meng QR, et al. Inhal Toxicol 2006; 18(8):555-568). Almost all of these models have concentrated on the induction of emphysema, but only mild disease has been induced (Churg A, Wright J L. Contrib Microbiol 2007; 14:113-125), with none of the models showing the smoke-independent progression seen in advanced human disease. There are also no established animal models of cigarette smoke-induced chronic bronchitis or acute exacerbations. The use of animal models for infection studies is complicated by the fact that there are species-specific differences in the resulting pathological changes (Xatzipsalti M, Papadopoulos N G. Contrib Microbiol 2007; 14:33-41) and mechanisms of infection (e.g. ICAM-1 required for cell binding of rhinoviruses (Staunton D E, et al. Cell 1989; 56(5):849-853) is not present on cells of most animals). Whilst human models of virus-induced exacerbations of asthma have been widely used (Papi A, et al. Curr Opin Pharmacol 2007; 7(3):259-265), apart from one study using the common cold virus (Mania P, Johnston SL. Chest 2006; 130(4):1203-1210), this approach has not been widely exploited in COPD. Researchers have been worried about the safety and ethics of provoking acute exacerbations in such patients who are often older and at risk of cardiovascular complications. Thus, application of the in vivo model is likely to remain difficult, in particular for proof of concept studies with novel anti-viral therapeutics.

Similarly, existing animal models of asthma exacerbation rely almost exclusively on ovalbumin sensitization followed by antigen challenge, a model which, although mimicking the human physiological effects of allergy to some degree, almost certainly does not follow the now widely recognised complex aetiology of a disease such as asthma.

Studies with human material have been pursued using simple cell cultures of bronchial epithelial cells. Epithelial primary and air liquid interface (ALI) systems have provided valuable insight into responses to viruses in asthma (Wark P A, J et al. J Exp Med 2005; 201(6):937-947) and COPD. However, these culture systems are unable to model the complexity of interactions between the epithelium, immune system, viruses and environmental stimuli of the airway (Wilkinson T M, et al. Chest 2006; 129(2):317-324).

An aim of the present invention is to provide an improved method to model disease and/or to develop drugs/therapy for a particular disease.

According to a first aspect of the invention, there is provided a method of tissue analysis comprising the steps of:
providing a tissue sample comprising cells of at least two different cell types;
exposing cells in the tissue sample to an agent ex vivo;
exposing the cells to three or more detection means arranged to distinguish between three or more different cell types and/or different cell states;
detecting the detection means in order to determine the different cell types and/or different cell states in the sample.

According to another aspect of the invention, there is provided a method of modelling a disease state comprising the steps of:
providing a tissue sample comprising cells of at least two different cell types;
exposing cells in the tissue sample to at least one disease causing agent ex vivo;
exposing the cells to three or more detection means arranged to distinguish between three or more different cell types and/or different cell states;
detecting the detection means in order to determine the different cell types and/or different cell states in the sample.

According to a further aspect of the invention, there is provided a method of drug development comprising the steps of:
(a)-providing a tissue sample comprising cells of at least two different cell types;
exposing cells in the tissue sample to a potential therapeutic agent ex vivo;
exposing the cells to three or more detection means arranged to distinguish between three or more different cell types and/or different cell states;
detecting the detection means in order to determine the different cell types and/or different cell states in the sample; or
(b)-providing a tissue sample comprising cells of at least two different cell types from an individual to whom a potential therapeutic agent has been administered;
exposing cells in the tissue sample to an agent ex vivo;
exposing the cells to three or more detection means arranged to distinguish between three or more different cell types and/or different cell states;
detecting the detection means in order to determine the different cell types and/or different cell states in the sample.

In step (b) the method may be used to ascertain the therapeutic efficacy of a potential therapeutic agent. Alternatively, the steps in part (b) may be performed using a known therapeutic agent rather than a potential therapeutic agent, again this may be to study the therapeutic efficacy of the therapeutic agent.

Preferably the detection means are detected by flow cytometry. Preferably the detection means are not detected by immunohistochemistry. Immunohistochemistry techniques can only be used to distinguish between two different cell types and/or states in a particular sample. Furthermore the use of flow cytometry is much more rapid and quantitative than immunohistochemistry.

The method of the invention may have a benefit in helping a researcher to examine and quantify changes to cells within complex tissue explants which mimic in vivo conditions and to model the effects of pharmacological interventions on disease. The method may also advantageously aid pre-clinical testing of novel therapeutic agents in disease relevant systems using human derived tissue without risk to individuals. The experimental results available from this system may also inform on novel disease mechanisms, the potential efficacy and toxicity of novel compounds prior to clinical testing and may accelerate and improve target and compound selection in the drug development process. In particular, this method may advantageously aid the identification of “responders” to treatment prior to human trials, and identification of those at risk or susceptible to disease, for example those with co-morbidities. The method may also allow testing of agents for individuals with relevant existing morbidity which would normally preclude their inclusion in clinical trials. The method may also act as a marker for statistically powering further clinical studies.

The tissue sample may comprise structural tissue, for example connective tissue, epithelial tissue, muscle tissue, or nervous tissue. It is understood that the term structural tissue refers to tissue comprising cells of a body that are non-circulatory, i.e. structural tissue does not include red blood cells. However the term structural tissue is not intended to exclude leukocytes that are residing within non-circulatory tissue. The tissue may comprise non-circulatory tissue. Preferably the tissue is not blood. The tissue may comprise one or more of the following tissue types: mucosal epithelial, tumour, fibrotic, glandular, endothelial, neural, renal, hepatic, muscle, cardiac, connective, pulmonary and dermal tissue. Preferably the tissue is human tissue. Preferably the tissue is a sample of human lung tissue.

The tissue sample may comprise cells of at least four different types, alternatively at least five different cell types or more.

Preferably the tissue sample is explant tissue. An advantage of using explant tissue (comprising at least three, four, five or more different cell types) in this method is that it more closely replicates the natural environment of the cells in the tissue, which typically comprises a mixture of cell types (including immune cells). The method of the invention may therefore provide a more reliable and accurate model of healthy and diseased tissue responses than methods using fewer cell types, such as methods using monocultures of cell lines.

The tissue sample may be a tissue sample taken from a healthy individual or a tissue sample taken from an individual with a particular disease. These may be obtained by biopsy collection or as a result of routine surgical procedures.

The tissue sample may comprise one or more of eukaryotic cells, prokaryotic cells and viruses. The tissue sample may comprise cells of one or more of the following type bacterial cells, such as extracellular and intracellular bacteria; yeast/fungal cells; and mammalian cells, such as epithelial cells, endothelial cells, fibroblast cells, muscle cells, goblet cells, fat cells, sensory cells, inflammatory/immune cells, such as lymphocytes (for example, T-cells, B-cells, natural killer cells) leukocytes (white cells), such as phagocytes (for example, macrophages, neutrophils, and dendritic cells), mast cells, eosinophils, and basophils, or combinations thereof.

The method of the invention advantageously allows a wide variety of different cell types to be studied in a single model, thus, closely replicating the in vivo environment for cells.

A cell state may comprise any of the group selected from an infected cell such as bacteria or virus infected cells; a non-infected cell; an activated/stimulated cell, for example activated by infection, an allergen/antigen, cell signalling or a cytokine; an immobilised cells (for example when stimulated/activated); a live cell; a dead cell; a dying cell; a dividing cell; a growing cell; a dormant cell; and a cell presenting a specified antigen. As an example, an infected cell is considered to have a different cell state than a non-infected cell, and a dead cell is considered to be in a different cell state than a live cell.

The determination of a variety of different cell states provides a benefit that complex disease, drug interaction, or infectious processes can be monitored in a complex environment.

Different cell states and/or cells types may be distinguishable by at least one detection means of the three or more detection means used in the method of the invention.

The detection means may be arranged to detect a specific cell marker which indicates the cell type or state. For example, the detection means may interact with or bind to molecules in the cell, or molecules presented on the cell surface, or molecules secreted by the cell. The detection means may interact or bind with a feature or body present in the cell or on the cell surface. For example, a detection means may bind to an intracellular microorganism in or on a cell and distinguish a cell in an infected state relative to a non-infected cell which has no intracellular microorganism infection. The presence, absence or abundance of a protein or molecule may determine a cell state. The expression of a nucleic acid sequence may determine a cell state. The presentation of an antigen on the cell surface may determine the cell state.

The method of the invention may use at least three, four, five, six, seven, eight, nine, ten or more different detection means. The method of the invention may use at least five different detection means.

An advantage of using at least three or more detection means is that several different cell types and/or several different cell states may be monitored in the same complex environment. Preferably the different detection means used can be detected simultaneously or substantially simultaneously. Preferably flow cytometry is used to detect the different detection means. By using a flow cytometer with multiple lasers of different wavelength multiple detection means can be analysed at once. By using different detection means, for example different flourophores that can be excited by the same or different wavelengths to emit different wavelengths of light, multiple parameters can be considered at once. For example, in one embodiment nine different colours are produced and analysed from multiple flourophores and three lasers. With a greater number of detection means (for example flourophores) and a greater number of means for detecting (for example lasers of different wavelength) more analysis can be done on different cell states and/or different cell types.

The detection means may be selected from antibodies, probes, such as PNA probes, molecular stains/dyes, and ligands, or combinations thereof. A detection means may comprise a live/dead stain. The detection means may be labelled, such as fluorescently labelled. The detection means may comprise a labelled antibody arranged to bind to an epitope which is characteristic of, a marker for, a particular cell type and/or cell state. Alternatively, or additionally, the detection means may comprise a labelled PNA probe comprising a sequence arranged to bind to a complimentary sequence present on and/or in a cell which is characteristic of, a marker for, a particular cell type and/or a cell state.

The detection means may comprise an antibody, preferably a monoclonal antibody, fluorescent dyes (e.g. propidium iodide, calcein-AM, mitotracker) or other types of fluorescently labelled probes (e.g. PNA, DNA, proteins). Targets for the detection means may include cell surface markers, intracellular proteins, cytoskeletal proteins and/or viral/bacterial proteins, and/or nucleotides, or combinations thereof.

The detection means may comprise an anti-lineage (Lin-1) cocktail monoclonal antibody, optionally tagged with fluorescein isothiocyanate (FITC).

The tissue sample may be dispersed/broken down by enzyme digestion or by physical dispersion. The tissue sample may be digested to form a cell suspension. The tissue sample may be digested using collagenase. The tissue sample may be digested with collagenase I, for example, derived from Clostridium histolyticum.

Preferably the tissue sample is exposed to the agent prior to dispersion. Preferably the tissue sample is intact when exposed to the agent.

An advantage of using a collagenase, such as collagenase I, is that it is very specific for collagen, thus, it does not interfere with the detection means, such as antibodies.

The agent to which the tissue sample is exposed may comprise a disease causing agent and/or a potential therapeutic agent. The agent may comprise any of the group selected from a virus; a phage; a bacteria; a fungus; a prion; a drug; a chemical; a nucleic acid, such as RNA or DNA; a protein; an antibody; an allergen; an oxidant; a hormone; a cell signalling molecule; and a toxin, such as nicotine; or combinations thereof. The agent may be a pre-cursor molecule, such as a prodrug.

Where the agent is a disease causing agent the agent may be one or more of a virus; a phage; a bacteria; a fungus; a prion; a drug; a chemical; a nucleic acid, such as RNA or DNA; a protein; an antibody; an allergen; an oxidant; a hormone; a cell signalling molecule; and a toxin, such as nicotine; or combinations thereof. The disease causing agent may be an infectious agent or a part of an infectious agent.

Where the agent is a potential therapeutic agent the agent may be a drug, a prodrug, a chemical, a nucleic acid, a protein or an antibody.

The method may further comprise contacting the tissue sample with an inhibitor. The inhibitor may be an inhibitor of the therapeutic agent, alternatively, an inhibitor of the disease causing agent. The inhibitor may comprise any of the group selected from a drug; an antiviral; an antibiotic; an antibody; a hormone; a nucleic acid, such as RNA or DNA; a protein; a phage, a cell signalling molecule; and a toxin; or combinations thereof. The inhibitor may be a pre-cursor molecule, such as a prodrug.

The agent and/or inhibitor may be provided in a carrier, such as a pharmaceutically effective carrier.

The method may further comprise detection and/or identification of material in the sample which is not live and is not dead, for example cellular debris or clumps of aggregated protein. This material may be detected and/or identified by the detection means, preferably it is detected by using a live/dead stain.

Detection and/or identification of material that is not live and not dead is advantageous because digestion of tissue liberates large fragments of structural proteins, such as elastin and collagen, which are large enough to be incorrectly registered as cells by a flow cytometer.

The tissue analysis may be for one or more of the following (i) analysing the effect of an agent on the tissue; (ii) modelling a disease process in a tissue when exposed to an infectious agent; (iii) pre-clinical toxicity testing of novel pharmacological agents; (iv) pre-clinical efficacy testing of novel pharmacological agents; (v) identification of individuals susceptible to infection; (vi) identification of individuals susceptible to disease; and (vii) identification of the mechanisms of susceptibility to disease.

The method of the invention may be used to model human lung conditions such as COPD and/or asthma ex vivo.

The method of the invention may be used to develop drugs to treat human lung conditions such as COPD and/or asthma.

The combination of using clinically relevant human tissue, relevant stimuli such as respiratory viruses to model disease and the ability to analyse individual components of complex tissue is particularly advantageous. The method of the invention used ex vivo with human bronchial tissue explant may advantageously enable detailed studies of microbial-induced exacerbations of COPD and asthma, and may help to develop therapy with novel agents which cannot be studied by existing techniques.

According to another aspect of the invention, there is provided a kit for tissue analysis comprising a plurality of detection means arranged to distinguish different cells and/or cell states in a tissue sample.

The kit may further comprise a tissue digesting agent, such as collagenase.

The kit may further comprise instructions for carrying out the method of the invention.

According to another aspect of the invention, there is provided the use of the method of the invention for analysing the effect of an agent on a tissue explant.

According to another aspect of the invention, there is provided the use of the method of the invention for modelling a disease process in a tissue sample when exposed to an infectious agent.

According to another aspect of the invention, there is provided the use of the method of the invention for pre-clinical toxicity and/or efficacy testing of one or more novel pharmacological agents.

According to another aspect of the invention, there is provided the use of the method of the invention for the identification of individuals susceptible to infection and/or disease.

According to another aspect of the invention, there is provided the use of the method of the invention for the identification of a mechanism of susceptibility to disease.

According to another aspect of the invention, there is provided the use of the method of the invention for the identification of individuals likely to respond to, or have an adverse reaction to, a particular therapy

It will be appreciated that optional features applicable to one aspect or embodiment of the invention can be used in any combination, and in any number. Moreover, they can also be used with any of the other aspects or embodiments of the invention in any combination and in any number. This includes, but is not limited to, the dependent claims from any claim being used as dependent claims for any other claim in the claims of this application.

An embodiment of the present invention will now be described herein, by way of example only, with reference to the following figures.

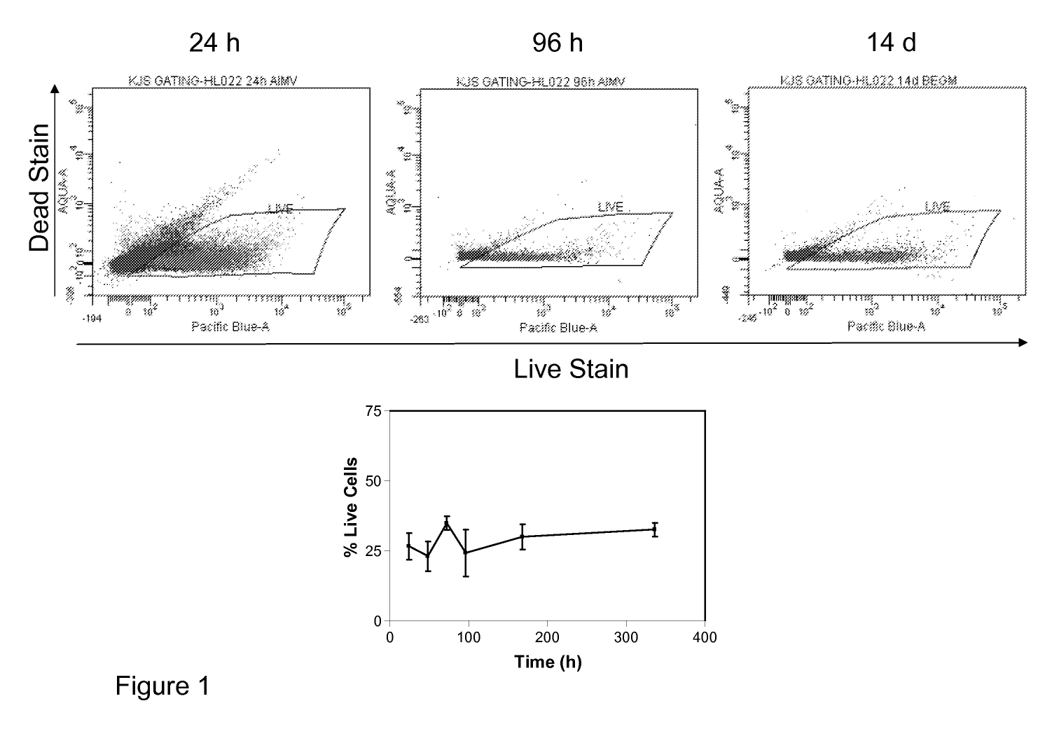

FIG. 1—shows the results of the flow cytometry analysis of tissue in culture;

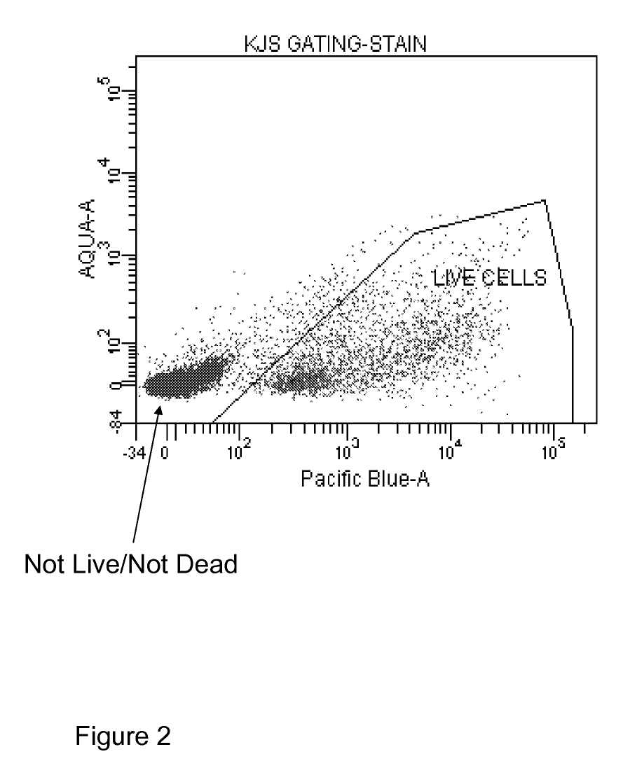

FIG. 2—shows the detection by flow cytometry of not live/not dead material from collagenase dispersed biopsy material;

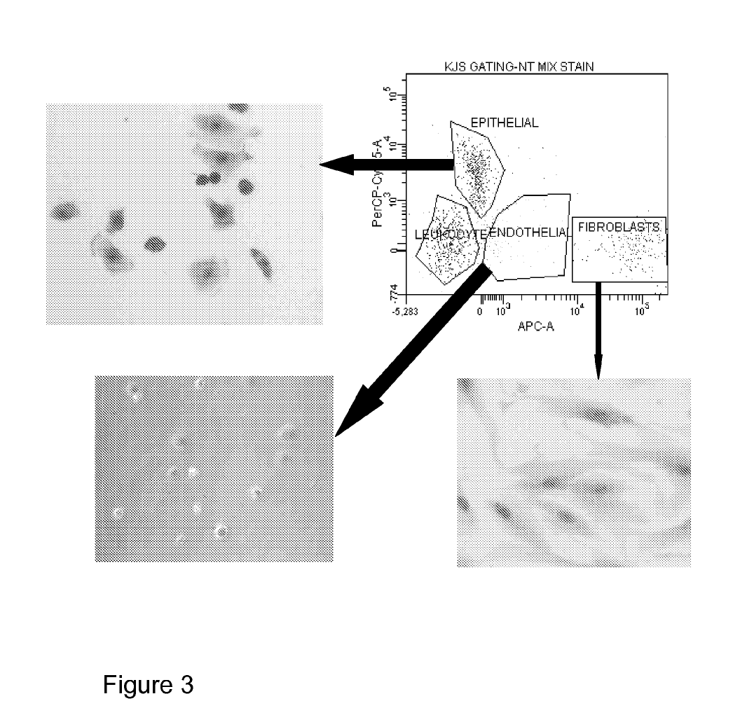

FIG. 3—illustrates the sorting of structural cells from a cultured cell mix by flow cytometry;

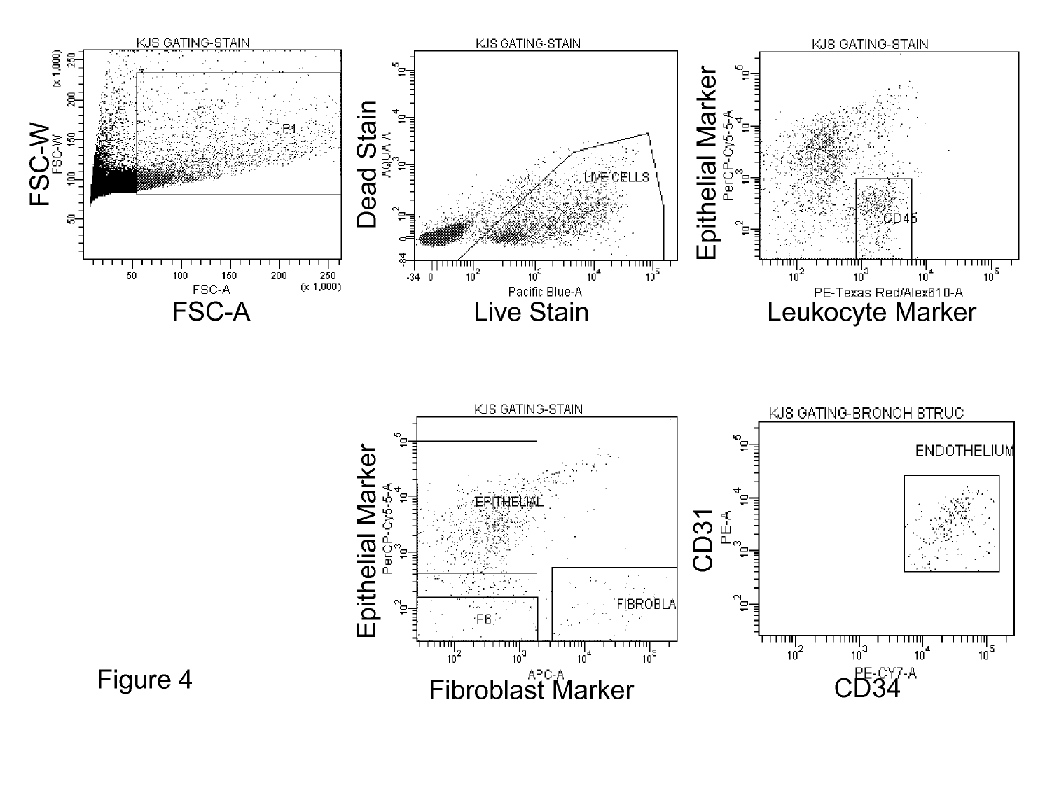

FIG. 4—shows the analysis of structural cells from collagenase digested lung tissue by flow cytometry;

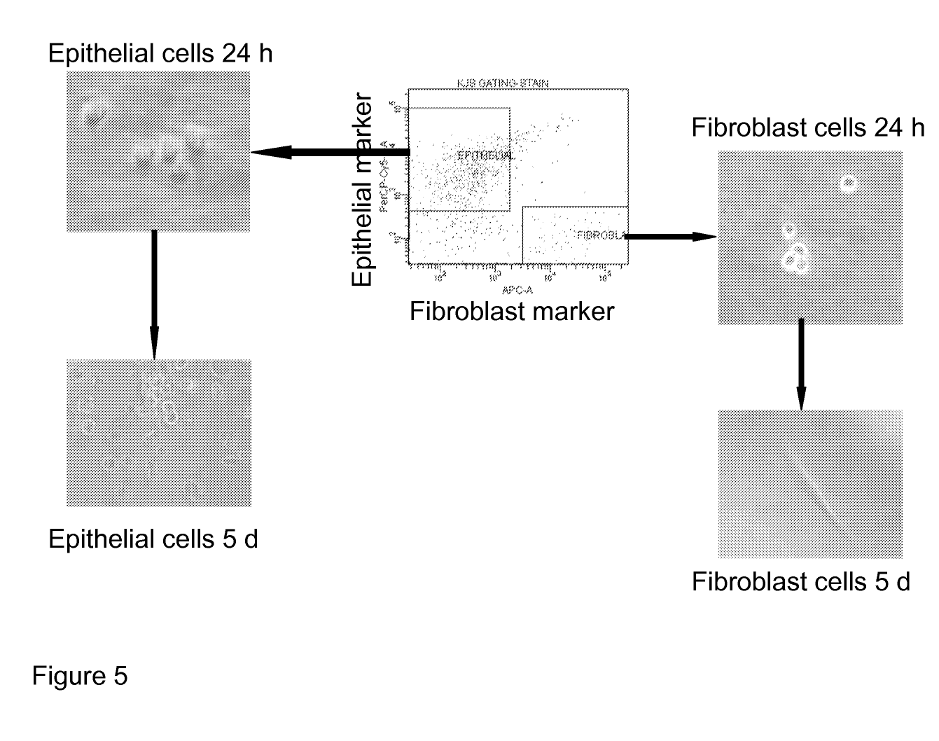

FIG. 5—shows the sorting of structural cells from collagenase digested lung tissue by flow cytometry;

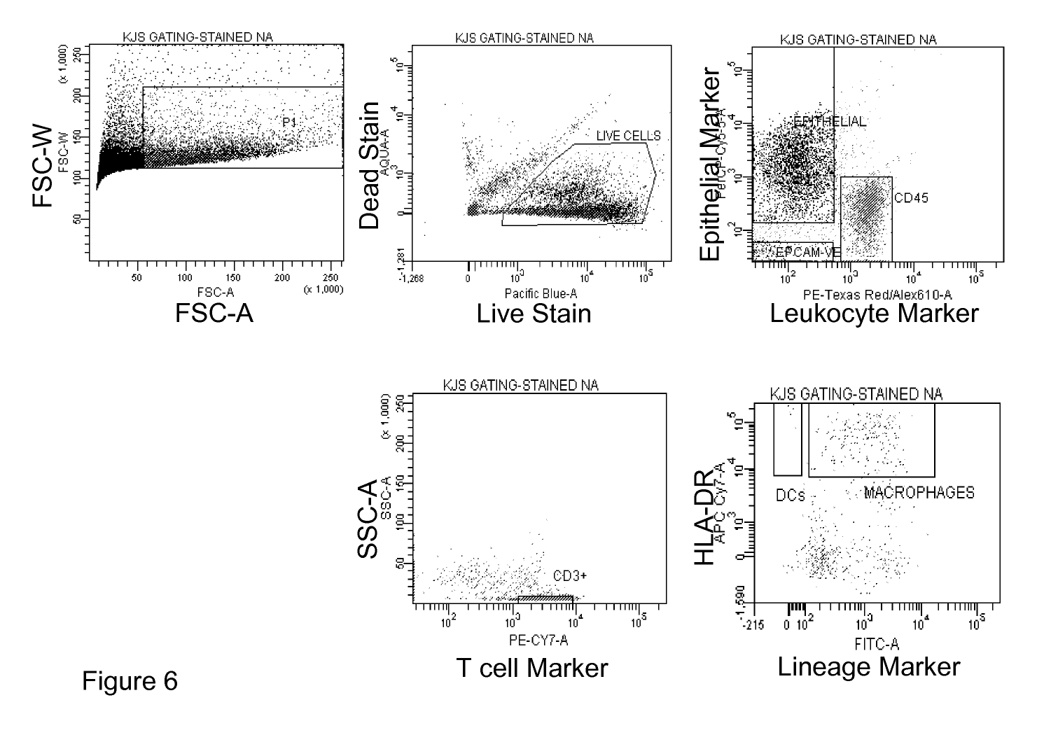

FIG. 6—shows the analysis of inflammatory cells from collagenase digested lung tissue by flow cytometry;

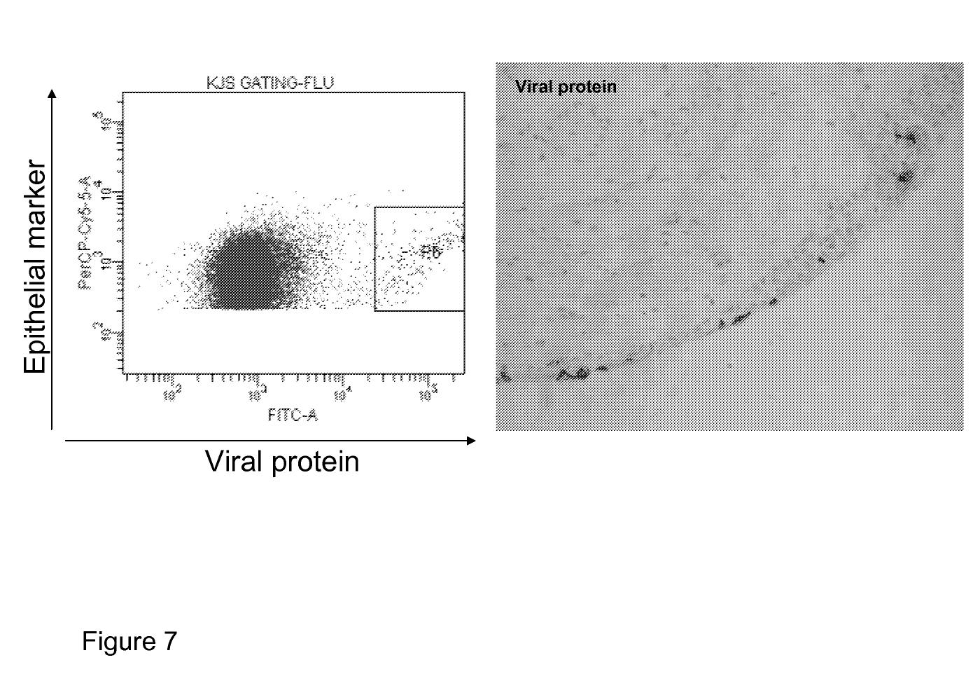

FIG. 7—illustrates a comparison of the analysis of a sample by flow cytometry and the analysis of a sample by immunohistochemistry;

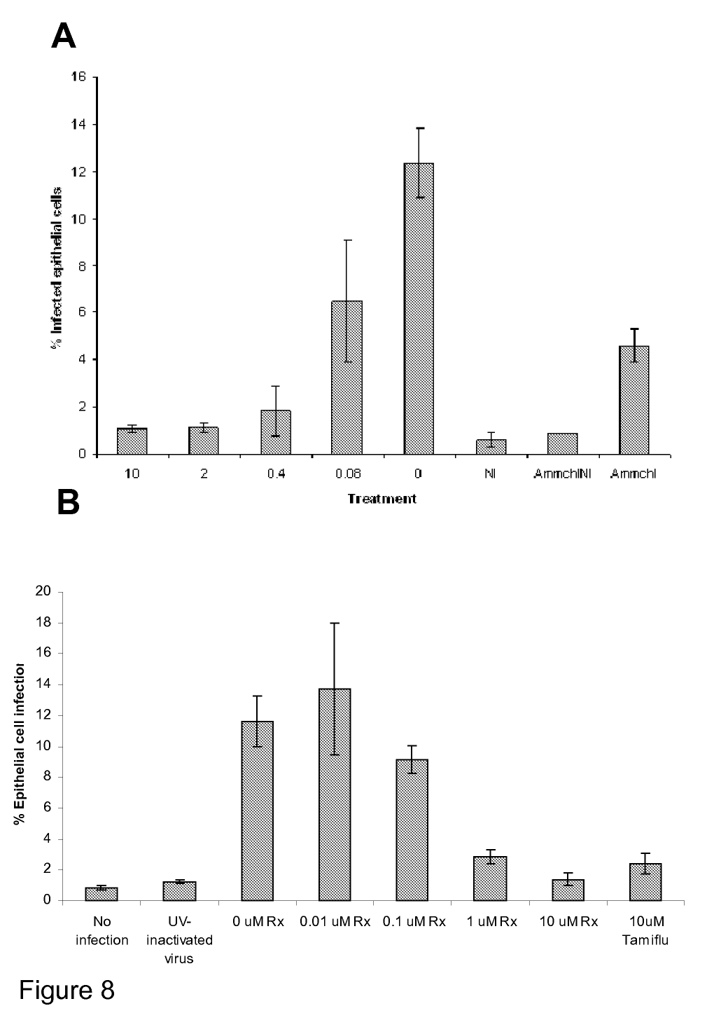

FIG. 8—illustrates a benchmarking model using (A) ammonium chloride and oseltamivir; (B) a novel anti-viral compound (Rx);

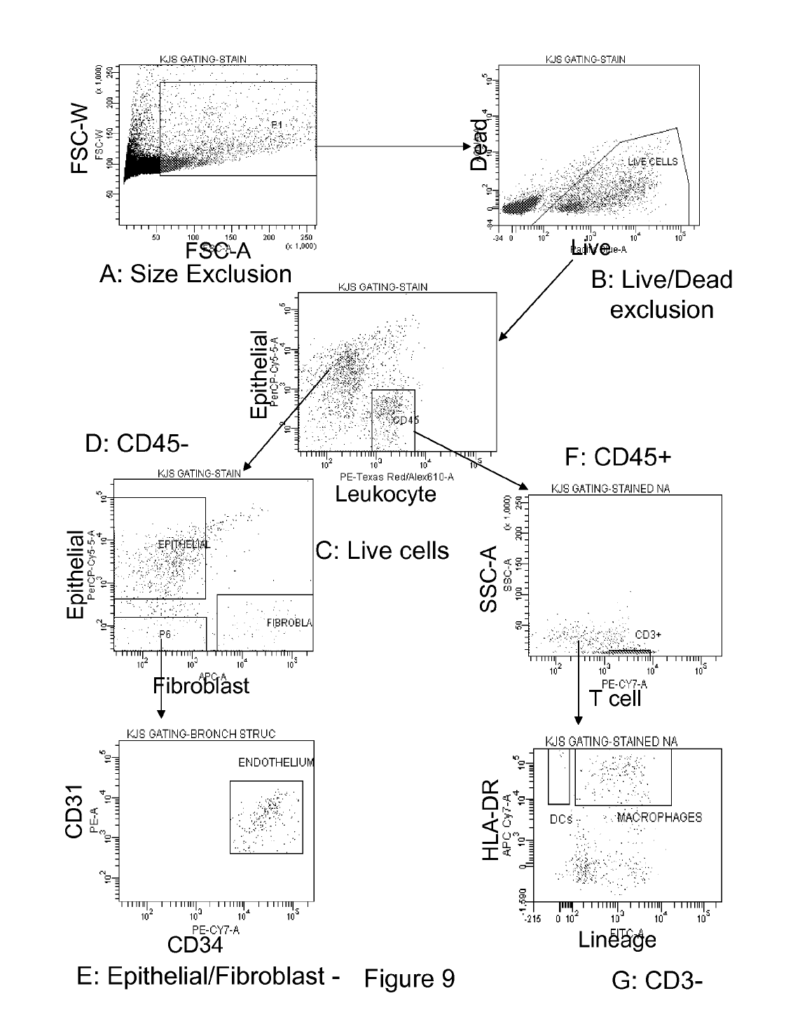

FIG. 9—shows the gating strategy on a flow cytometer used to identify the different cell types in the tissue;

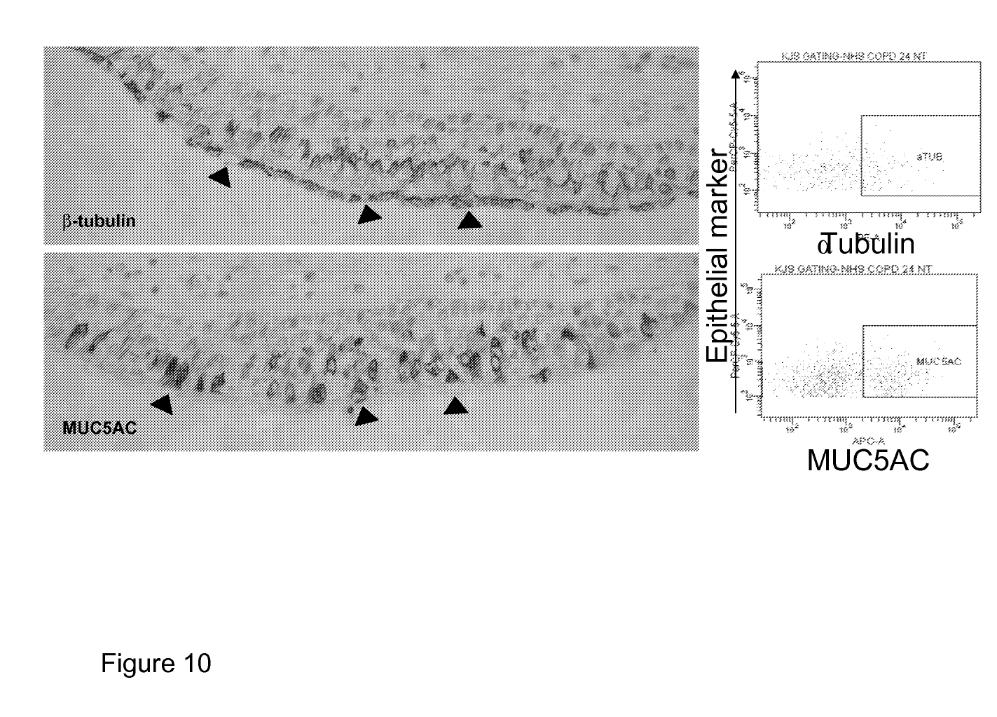

FIG. 10—shows the detection, by immunochemistry and flow cytometry, of different epithelial cell phenotypes in a collagenase digested bronchial lung tissue sample; and

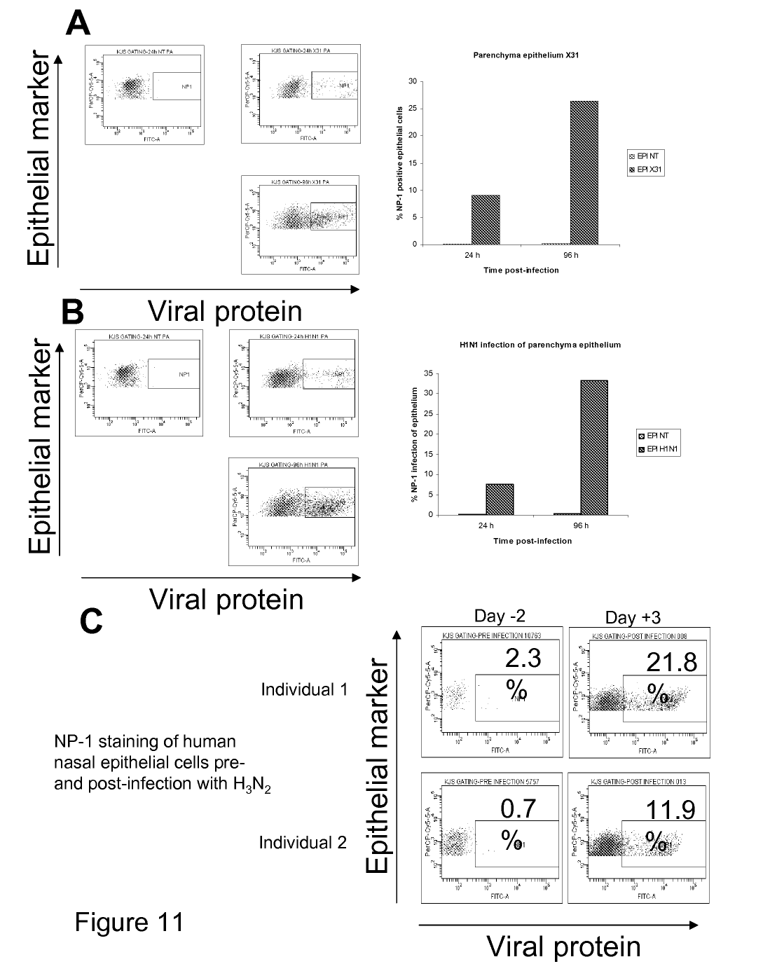

FIG. 11—illustrates an increase in infection over time using (A) X31 and (B) circulating strain of H1N1 (A/New Caledonia/20/99). (C) Detection of circulating strain of H3N2 in nasal epithelia of healthy volunteers 3 d after infection with virus.

### MATERIALS

Virus stocks (stored at −80° C.)

Virkon—From Fisher Scientific

Bronchial biopsies were obtained by flexible fibreoptic bronchoscopy from both healthy controls and asthmatic subjects.

Explant tissue was cultured in freshly prepared AIM-V media supplemented with 50 IU/ml penicillin, 50 mg/ml streptomycin, 2 mM L-glutamine, 1 mM Sodium Pyruvate, 0.5 μg/ml Amphotericin B and 0.004% (v/v) 2-mercaptoethanol (All Invitrogen).

Tissue was digested in unsupplemented phenol-red free RPMI media (referred to in method as basal RPMI—Lonza) containing 1 mg/ml (w/v) collagenase I from Clostridium histolyticum (Sigma)).

Cells derived from digested tissue were incubated with antibody in FACS buffer (0.5% (w/v) Bovine serum albumin (BSA), 2 mM EDTA in Dulbecco's phosphate buffered saline without magnesium or calcium (DPBS—all reagents Sigma)).

Cells derived from digested tissue were fixed and permeabilised for intracellular staining using the Becton Dickinson (BD) Cytofix/Cytoperm Fixation/Permeabilisation kit. BD Perm/Wash from this kit was diluted to 1×working solution using FACS buffer.

### Ex Vivo Modelling of Therapeutic Interventions in Airways Disease

Bronchial and parenchymal lung tissue samples were obtained and studied initially to ensure that the explant tissue could be cultured without significant loss of cellular material or cellular function. Live/Dead stain (purchased from Invitrogen) was used to assess the number of viable cells. There was no significant loss of cell numbers after 14 days in culture (FIG. 1). Furthermore, functional cilia on the bronchial samples could still be observed after 14 days. Thus the tissue can be cultured in these conditions for up to two weeks without significant loss of function or viable cells.

A Live/Dead stain, such as that available from Invitrogen, was used to identify and eliminate any not live and not dead (FIG. 2). This was important as collagenase disgestion of lung tissue also liberates large fragments of structural proteins (e.g. elastin and collagen) which are large enough to be registered as cells by the flow cytometer. In addition these fragments of protein have autofluorescence properties when excited by the lasers used by flow cytometers and can also non-specifically bind fluorescently labelled antibodies. This can substantially complicate analysis. Thus the ability to remove such events increases the specificity of the analysis from lung tissue.

Having demonstrated that cells within the explant can be distinguished using a live/dead stain, fluorescently-labelled antibodies for use in the discrimination of different structural and inflammatory cell types within the explant were obtained and analysed. To validate and optimise the antibodies, the cultured cells derived from human tissue were used to assess the expression of cell surface markers using different antibodies with the goal of being able to discriminate each cell type with a different antibody, whilst each antibody is labelled with a fluorochrome which does not overlap in spectra with another. All flow cytometry gating was set using an appropriate isotype control antibody. The following cell types have been studied, and the following antibodies have been titrated for use with each cell type:

To validate the method five cell types were analysed by flow cytometry using a cell specific antibody.
1. Cell type: Primary bronchial epithelial cells (PBECs) derived from bronchial brushings—Antibody: EpCAM PerCPCy5.5 (CD326-BD(Becton Dickinson).
2. Cell type: Primary human lung fibroblasts outgrown from bronchial biopsies—Antibody: CD90 APC (BD).
3. Cell type: Primary human endothelial cells derived from umbilical cord (HUVECs)—Antibody: CD31 PE and CD34 PECy7 (both BD).
4. Cell type: Primary human airway smooth muscle derived from enzymatically digested bronchial tissue—Antibody: CD90 APC.
5. Cell type: Human peripheral blood mononuclear cells (PBMCs)—Antibody: CD45 PE-AlexaFluor 610—Invitrogen.

Once these cell types were distinguished individually, these cell types were mixed together in proportions based on previous biopsy analysis in order to mimic lung tissue. To further mimic debris and dead cells, a proportion of cells of each type were killed by exposure to hydrogen peroxide (60% PBECs, 20% PBMCs, 10% fibroblasts, 10% HUVECS) for inclusion in the mixture. Cells were then sorted back into culture to ensure the purity of the analysis (FIG. 3).

Having validated the method using a known mix of cell types, the method was applied to an explant of lung tissue.

Prior to applying the antibodies to the lung tissue, it was ensured that none of the epitopes recognised by the antibodies would be adversely affected by collagenase digestion by incubating the cell mix described above in collagenase (1 mg/ml) at 37° C. for 1.5 h with stirring. The surface marker expression was then assessed by flow cytometry. None of the markers described herein were affected by collagenase treatment.

Once the specificity and robustness of the antibodies were assured the full panel of antibodies were applied to collagenase dispersed bronchial tissue (FIG. 4) and the cells were sorted. It was observed that antibody specificity was still retained in the real tissue environment (FIG. 5). In addition it was demonstrated that the samples could be split so that the leukocytes present in the CD45 gate could also be identified (FIG. 6). Antibodies/markers used are:
1. CD3 PECy7 for identification of T cells.
2. HLA-DR APCCy7 for identification of macrophages, dendritic cells and T cell activation.
3. Lin-1 FITC—cells negative for this marker are dendritic cells
4. CD25 PE for T cell activation.
5. CD8 APC for identification of CD8+ T cells and negative selection of CD4+ T cells.

Once all the relevant cell types could be identified within lung tissue the tissue was then challenged with an infectious agent to determine whether it could be detected using the flow cytometric methods previously described, and more importantly whether treatment of the tissues with therapeutic agents currently in use would affect the observed outputs. A laboratory strain of influenza virus H₃N₂X31 was used to infect the tissue and the cell types infected were traced using an antibody raised against the nucleoprotein (NP-1) of this virus. Before exposing the lung tissue to virus, the amount of fluorescently labelled NP-1 antibody to use was optimised by infecting PBEC cultures with X31 virus and quantifying the amount of infection observed by flow cytometry. An added complication was introduced as NP-1 is expressed intracellulary, thus the cells were fixed and permeabilised using BD Cytofix/Cytoperm kit so that the antibody could detect the viral protein. At the same time it was checked that this procedure did not interfere with the selected antibody panel, either in cultured primary cells or on lung tissue.

To ascertain the initial infection patterns of this virus in lung tissue the infected tissue was embedded in glycol methacrylate (GMA) resin and the tissue was analysed by immunohistochemistry using an unlabelled NP-1 antibody. Surprisingly, only epithelial cells and macrophages in the tissue were infected upon exposure to virus and there was no evidence of spill-over into any other cell type after 24 hours. The immunohistochemistry data was then compared to the data obtained using the flow cytometer and it was found that the techniques were comparable in the data obtained (FIG. 7), but advantageously, the flow cytometry data was more rapid and more easily quantifiable.

To ensure that real infection was being observed and not just binding of the virus particle to cells a portion of X31 preparation was UV-inactivated and used as a negative control. Dose response and time course experiments up to 96 hours with the virus were then performed to optimise the system of detection in both bronchial and parenchymal tissue. The primary output of these investigations has been the percentage of cells infected, but additional readouts that have been optimised include release of viral particles into cell culture supernatant and an upregulation of HLA-DR expression on infected epithelial cells.

To ensure that this system is also suitable for drug testing a non-specific inhibitor of viral infection, namely ammonium chloride, and a neuraminidase inhibitor, namely oseltamivir, were used. This model demonstrated the efficacy of both compounds (FIG. 8). Furthermore, the use of this method has been validated for detection of circulating influenza strains of both H₁N₁ and H₃N₂ types. (FIG. 11).

This work was conducted using both bronchial and parenchymal tissue demonstrating that this technique can be applied from all lung-derived material.

The method provides a novel, rapid and quantitative method for investigating airways disease, the mechanisms of airway infection and the efficacy of novel therapeutic compounds.

### Method for Infection and Analysis of Resected Lung Tissue Method

1. Collect the tissue sample at room temperature in sterile 60 ml pots.
2. Transfer the tissue sample to a 10 cm petri dish and dissect into 2 mm³ pieces.
3. Quickly transfer the pieces to a 6-well plate containing 4 ml DPBS-per well.
4. When tissue sample is completely dissected, wash all the pieces by completely removing the DPBS and replacing with basal RPMI. Repeat this process to remove blood.
5. Put 1 ml basal RPMI into each well of a 24-well plate.
6. Transfer the tissue sample pieces in pairs into each well of the 24-well plate using a cut-off 1 ml pipette tip.
7. Completely remove the RPMI and replace with 540 μl of AIM-V medium.
8. Add 60 μl of 10× inhibitor stock and incubate for 2 h at 37° C.
9. Remove culture medium and wash tissue sample pieces with 1 ml of basal RPMI.
10. Add 520 μl of AIM-V medium and 60 μl of inhibitor.
11. Add 20 μl of X31 virus or UV-inactivated X31. Incubate for a further 2 h at 37° C.
12. Completely remove culture medium and wash tissue sample pieces with 1 ml basal RPMI. Repeat once.
13. Add 540 μl AIM-V medium and 60 μl inhibitor and incubate for a further 20 hours.
14. If culturing for longer then, repeat steps 12 & 13 every 24 hours.
15. Sample the culture medium by removing 200 μl for viral shedding assay.
16. Collect tissue sample pieces and transfer into 5 ml aliquots of digestion buffer in a 25 ml universal tube containing a magnetic flea.
17. Incubate at 37° C. for 90 min with agitation.
18. Strain mixture through a 100 μm filter using a luer-lock 2.5 ml syringe into a polypropylene FACS tube.
19. Centrifuge cells at 400 g for 5 minutes at 4° C. Discard supernatant and resuspend the cells in 100 μl of FACS buffer containing 2 mg/ml IgG from human serum.
20. Stain the cells for surface markers using the following proportions per sample:
   10 μl EpCAM PerCPCy5.5
   10 μl CD45 PE AF610

For analysis of structural cells add:
5 μl HLADR APCCy7
5 μl CD31 PE
5 μl CD34 PECy7
2.5 μl 1:10 dilution of CD90 APC

For analysis of inflammatory cells add:
5 μl HLADR APCCy7
5 μl CD8 APC
5 μl CD25 PE
2.5 μl CD3 PECy7
21. Incubate for 30 minutes on ice and then add 2 ml of FACS buffer. Centrifuge at 400 g for 5 min at RT.
22. Discard supernatant and resuspend cells in 200 μl FIX/PERM. Incubate for 20 min on ice in the dark.
23. Add 2 ml of PermWash and centrifuge at 400 g for 5 min at RT.
24. Resuspend the cells in 100 μl PermWash and add 1 μl NP-1 AF488 antibody. Incubate for 30 min on ice in the dark.
25. Add 2 ml of PermWash and centrifuge at 400 g for 5 min at RT.
26. Discard supernatant and resuspend cells in 500 μl FACS buffer.
27. Store at 4° C. in the dark until analysis. Strain through FACS filters before applying to the FACSAria.
28. Set gates on FACSAria using unstained control and isotype control tubes (see FIG. 9).
29. Quantify the number of NP-1+ve cells.

### SUMMARY

The method described herein provides a drug development platform for use in the identification and testing of anti-inflammatory, anti-bacterial and anti-viral compounds, in particular, compounds that have not yet been licensed for use in humans to provide proof of concept data for Phase I trials. Furthermore, the method is not limited to influenza or just viral infection of the airways. As long as specific fluorescently-labelled detection methods exist (e.g. antibodies or PNA probes), the model can be extended to other viruses as well as bacteria and to other tissues. In addition, the method is also well suited to discovering novel mechanisms that underlie chronic diseases, such as asthma and COPD in the case of respiratory diseases, and to test therapeutics aimed at these novel targets. The method can also be used to assess drug toxicity, either via Live/Dead staining, caspase activation assays or by examining the tissue-conditioned media.

The method can not only be used for drug testing but may also be used to ascertain individual susceptibility to a virus. Viral infection of the pulmonary epithelium is patchy unlike infection of epithelial cells in culture (FIG. 8) and it could be that different cell types have different susceptibility to infection. By using labelled antibodies against, for example, goblet cells (MUC5AC), ciliated epithelium (acetylated a-tubulin) and basal epithelial cells (CD151) the method may be used to discriminate whether any particular cell type is the main target of initial infection (FIG. 10). Additionally different strains of influenza are thought to bind to cells expressing certain sialic acid isotopes. The method of the invention may use of fluorescently labelled lectins which have specific binding properties for these sialic acids to pursue whether their expression is related to susceptibility to infection. This technique may be broadened to other micro-organisms such as rhinovirus which can bind the cell surface marker CD54 (ICAM-1).

This extensively validated ex vivo human bronchial tissue explant model described herein enables detailed studies of microbial-induced exacerbations of COPD and asthma, and may help to develop therapy with novel agents which cannot be studied by existing techniques

The majority of cell types present in the mucosa can be analysed with a higher throughput than conventional methods and this technique has vastly improved the ability to quantify cellular processes in the bronchi. Furthermore the method may enable ex vivo study of virus-induced airway inflammation that is associated with exacerbations of COPD and asthma. The method improves on both animal and in vitro systems in that the tissue is already disease specific.

To date the use of human tissue has been limited to simple cell culture systems which do not reflect the complexity of human diseases. The method of the invention provides valid human models of disease to validate host targets for a novel approach to developing treatments, such as anti-viral drugs.

1. A method of tissue analysis comprising the steps of:
providing a tissue sample comprising cells of at least two different cell types;
exposing cells in the tissue sample to an agent ex vivo;
exposing the cells to three or more detection means arranged to distinguish between three or more different cell types and/or different cell states;
detecting the detection means in order to determine the different cell types and/or different cell states in the sample.

2-33. (canceled)

34. The method of claim 1, wherein the method is used for modelling a disease state and the agent to which the cells in the tissue sample are exposed comprises at least one disease causing agent.

35. The method of claim 1, wherein the detection means are detected by flow cytometry.

36. The method of claim 1, wherein the tissue sample comprises structural tissue.

37. The method of claim 1, wherein the tissue sample comprises non-circulatory tissue.

38. The method of claim 1, wherein the tissue sample is explant tissue or a culture derived from explant tissue.

39. The method of claim 1, wherein the tissue sample comprises cells of one or more of the following type: bacterial cells, yeast cells, fungal cells, or mammalian cells.

40. The method of claim 1, wherein a cell state comprises any of the group selected from an infected cell; a non-infected cell; an activated/stimulated cell; an immobilised cell; a live cell; a dead cell; a dying cell; a dividing cell; a growing cell; a dormant cell; a cell presenting a specified antigen; and combinations thereof.

41. The method of claim 1, wherein the detection means is arranged to detect a specific cell marker which indicates the cell type or state.

42. The method of claim 1, wherein the detection means interacts with or binds to molecules in the cell, or molecules presented on the cell surface, or molecules secreted by the cell.

43. The method of claim 1, wherein the detection means comprises a live/dead stain.

44. The method of claim 1, wherein a target for the detection means includes cell surface markers, intracellular proteins, cytoskeletal proteins and/or viral/bacterial proteins, and/or nucleotides, or combinations thereof.

45. The method of claim 1, wherein the tissue sample is dispersed/broken down by enzyme digestion or by physical dispersion.

46. The method of claim 1, wherein the tissue is digested to form a cell suspension, optionally using collagenase.

47. The method of claim 46, wherein the tissue is digested to form a cell suspension using collagenase.

48. The method of claim 1, wherein the agent to which the cells are exposed comprises an infectious agent and/or a potential therapeutic agent.

49. The method of claim 1, wherein the method further comprises the step of contacting the cells with an inhibitor.

50. The method of claim 1, wherein the method further comprises the step of detection and/or identification of material in the sample which is not live and is not dead.

51. The method of claim 1, wherein the tissue analysis is for one or more of the following (i) analysing the effect of an agent on the tissue sample; (ii) modelling a disease process of the tissue sample when exposed to infectious agents; (iii) pre-clinical toxicity testing of novel pharmacological agents; (iv) pre-clinical efficacy testing of novel pharmacological agents; (v) identification of individuals susceptible to infection; (vi) identification of individuals susceptible to disease; or (vii) identification of the mechanisms of susceptibility to disease.

52. A kit for tissue analysis comprising a plurality of detection means arranged to distinguish different cells and/or cell states in a tissue sample.

53. The method of claim 1 wherein the method is used for one or more of (i) analysing the effect of an agent on a tissue explant; (ii) modelling a disease process in a tissue sample when exposed to an infectious agent; (iii) pre-clinical toxicity and/or efficacy testing of one or more novel pharmacological agents; (iv) the identification of individuals susceptible to infection and/or disease; (v) the identification of individuals likely to respond to, or have an adverse reaction to, a particular therapy: or (vi) the identification of a mechanism of susceptibility to disease.
